(19) United States
(12) Patent Application Publication
(10) Pub. No.: US 2015/0335817 A1
(43) Pub. Date: Nov. 26, 2015

(54) PUMP APPARATUS, SYSTEM AND METHOD OF USE

(71) Applicant: BIO HEALTH FRONTIERS, INCORPORATED, LAS VEGAS, NV (US)

(72) Inventor: PAUL LAMBERT, EL DORADO HILLS, CA (US)

(21) Appl. No.: 14/721,653

(22) Filed: May 26, 2015

Related U.S. Application Data
(60) Provisional application No. 62/002,818, filed on May 24, 2014.

(51) Int. Cl. A61M 5/148

(52) U.S. Cl. CPC A61M 5/1483

(57) ABSTRACT

A system and method of delivering and administering fluids used for infusion and similar therapies is provided that uses a reusable pump including a mechanism that eliminates contact between the pump material and the therapeutic fluid. An adapter for the pump, which is configured to interface with standard or customized drug reservoirs, is additionally provided.

### CROSS-REFERENCE TO RELATED APPLICATIONS

The present application claims priority from co-pending Provisional Patent Application No. 62/002,818, entitled “Improved Elastomeric Pump Apparatus, System and Method of Use and filed on May 24, 2014; that application being incorporated herein, by reference, in its entirety.

### BACKGROUND OF THE INVENTION

1. Field of the Invention

The invention relates to an improved pump apparatus, and more particularly, to an apparatus, system and method of delivering fluids used for infusion and similar therapies using an improved pump.

2. Description of the Related Art

In the field of medical devices used in infusion and similar therapies, medicinal or other fluidic treatments are delivered from a fluidic source via tubular lines to treatment areas within a patient via one or more needles or catheters inserted in the patient. In such medical devices, the tubular lines are interconnected via various connector assemblies such as luer connectors and other components. When fluidic therapy is delivered to a patient, a fluidic source and the means to deliver the fluidic treatment to the patient are required. Fluids may be sourced by a syringe and pump, a hanging bag, or other fluidic source. The fluidic delivery means may comprise a tubing set comprised of one or more tubular lines connects the fluid source (i.e., the proximal end) to the patient (i.e., the distal end). At the proximal end, tubular lines are terminated by connectors (usually female luer connectors) to permit their connection to the source of the fluid. At the distal end of the tubular lines, needles (such as subcutaneous, intramuscular, intravenous, epidural/spinal or similar types) deliver the fluidic treatment to the patient's treatment areas.

Fluids may be sourced by a syringe, a hanging bag, or other fluidic source, which are impelled by a variety of means out of the distal end of the fluidic source into the tubing set or other fluidic delivery means. Such impelling mechanisms include an electrical or mechanical pump, or even gravity.

Multiple pumps have been developed for the controlled infusion of pharmaceuticals over the last few decades. The state-of-the art has rapidly advanced and infusion pumps have been gradually improved relative to accuracy, user friendliness and ergonomics among other performance parameters. Infusion pumps can be categorized into ambulatory and hospital based devices. In terms of principle of operation, pumps can generally be electronic or mechanical devices. While electronic pumps have generally more accuracy than mechanical pumps, the latter offer sufficient control of drug delivery at a significant operation cost savings.

Elastomeric pumps are mechanical pumps based on the ability of a balloon to produce a relatively constant force as it deflates. Elastomeric devices are based on a flexible balloon (elastomer) that, when inflated exerts pressure during deflation that results in a relatively constant flow rate controlled by a microbore tubing connected between the elastomer and the patient port/connector. The balloon is filled with a specific pharmaceutical fluid which is gently pushed into the patient via a small diameter tubing set that connects the elastomer to the patient, thus controlling the flow rate. One such pump is illustrated in U.S. Pat. No. 7,322,961 to Forrest, that patent being incorporated herein in its entirety.

Elastomeric devices have been developed in various form factors as well as outer shell materials including hard and soft enclosures. Typical devices have a soft long shape that optimizes the form factor of the elastomer-pump/drug-reservoir combination. Outer shells are typically designed to match the overall geometry to serve as a case for the system device to be transported easily.

The nature of pharmaceutical products and their chemical interaction with plastics is an important factor in the design of drug delivery platforms. Regulatory agencies around the world are raising thresholds of drug-reservoir compatibility as more in-depth toxicological ramifications of leachables related to drug reservoirs become better understood.

In addition, pharmaceutical companies are continuously developing new drugs that demand increasingly more inert reservoirs, as these drugs' chemical compositions make them more prone to chemical reactions with not only the containers in which they are packaged but also those used to transfer and then deliver them at the clinical site.

Because known elastomeric devices make contact with the drug, they are discarded after use. What is needed is an elastomeric device that can be reused, so as to optimize the cost of such an elastomeric device.

### BRIEF SUMMARY OF THE INVENTION

It is accordingly an object of the invention to provide a system and method of delivering fluids used for infusion and similar therapies using an improved elastomeric pump. In one particular embodiment, an elastomeric pump is provided that eliminates drug-reservoir chemical incompatibility issues, or leaching of the container material into the drug.

In another embodiment of the invention, a pump is provided having mechanical/geometrical compatibility with multiple factory-shipped drug reservoirs without requiring the transfer of medicinal fluid from the factory-shipped reservoir into a delivery reservoir, thus simplifying the transfer procedures and manipulation.

In one embodiment, a cost benefit is provided based on the re-use of elastomeric components. In another embodiment, the size of the pump delivery system is optimized.

As used herein, the term “or” may be construed in either an inclusive or exclusive sense. Similarly, the term “exemplary” is construed merely to mean an example of something or an exemplar and not necessarily a preferred or ideal means of accomplishing a goal. Additionally, although various exemplary embodiments discussed below focus on verification of experts, the embodiments are given merely for clarity and disclosure. Alternative embodiments may employ other systems and methods and are considered as being within the scope of the present invention.

Reference in the specification to “one embodiment”, “one particular embodiment”, “an embodiment”, “another embodiment” or “another particular embodiment” means that a particular feature, structure, or characteristic described in connection with the embodiment is included in at least one embodiment of the invention. The appearances of the phrase “in one embodiment” in various places in the specification are not necessarily all referring to the same embodiment.

The present invention may address one or more of the problems and deficiencies of the prior art discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

Although the invention is illustrated and described herein as embodied in a pump apparatus, system and method of use, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

For the purpose of illustrating the invention, there is shown in the drawings an exemplary embodiment that is presently preferred, it being understood however, that the invention is not limited to the specific methods and instrumentality's disclosed. Additionally, like reference numerals represent like items throughout the drawings. In the drawings:

FIG. 1 is a block diagram of a system for delivering a medicine or drug to a patient in accordance with one particular embodiment of the present invention;

FIG. 2 is a cut-away view of an elastomeric pump system in accordance with one particular embodiment of the invention; and

FIG. 3 is a cut-away view of an elastomeric pump system in accordance with another particular embodiment of the invention.

### DETAILED DESCRIPTION OF THE INVENTION

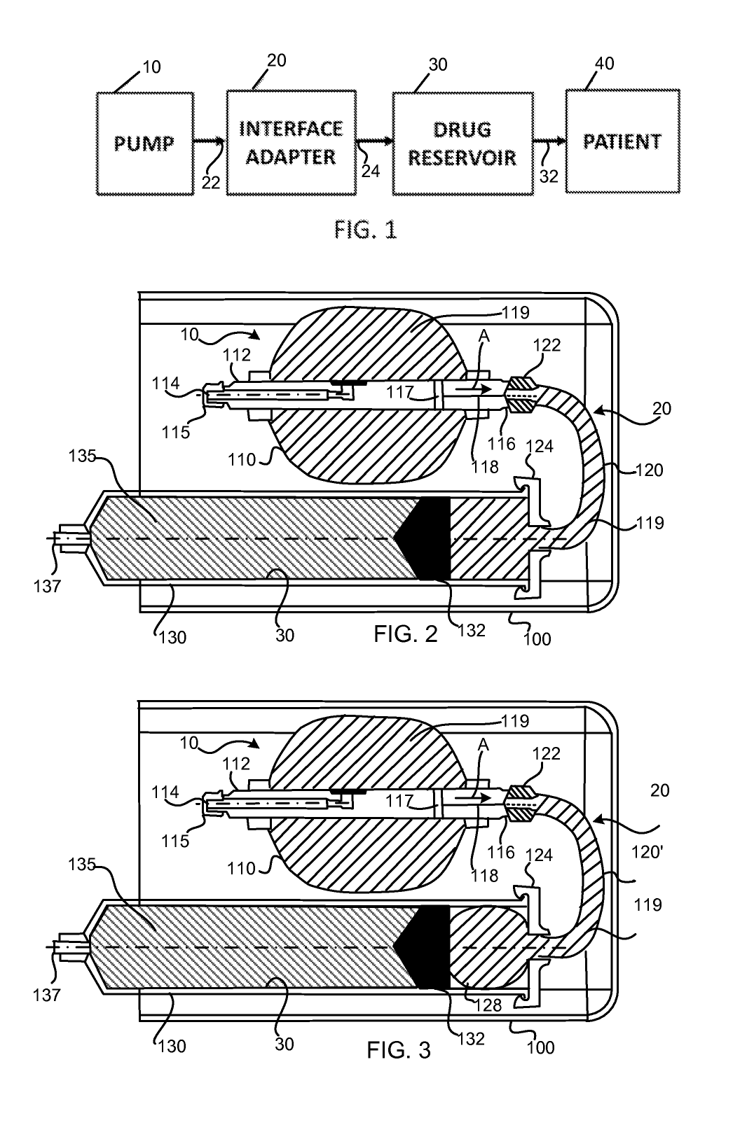

Referring now to FIG. 1, there is shown a block diagram of a system for delivering a medicine or drug to a patient 40 in accordance with one particular embodiment of the present invention. More particularly, a pump 10 is used to drive a medicinal fluid from a drug reservoir 30, via an interface adapter 20. In one particular embodiment, the pump 10 is an elastomeric device that is inflated with water or another impelling fluid, but the impelling fluid is fluidly isolated from the medicinal fluid contained within the drug reservoir 30.

In one particular embodiment of the invention, the interface adapter 20 is used to establish a fluid-tight seal and impelling chamber that connects the pump 10 at one end 22 of the interface adapter 20 with the drug reservoir at the other end 24 of the interface adapter 20. In one particular embodiment, a fluid-tight impelling chamber of the interface adapter 20 contains the impelling fluid as the pump 10 discharges the impelling fluid into the interface adapter 20 under pressure. Within the impelling chamber of the interface adapter 20, as the impelling fluid's volume and pressure increase, it pushes against the proximal end of the lumen of the drug reservoir 30 and forces the medicinal fluid out of the distal end of the drug reservoir 30 and into a tubing set or other delivery device 32, from which it is delivered into the patient 40. In this way, the pressurized impelling fluid becomes an engine that drives the medicinal fluid out of the drug reservoir 30 and into the patient 40.

Referring now to FIGS. 1 and 2, a system for delivering a medicine or drug to a patient in accordance with one particular embodiment of the invention will now be described. A housing 100 is provided containing elements corresponding to the pump 10, interface adapter 20 and drug reservoir 30. More particularly, an infusion pump 10 is provided to hold a pressurized fluid, which in accordance with the present invention is an impelling fluid, rather than a drug or medicine. In one particular embodiment, the impelling fluid is water, but may be another impelling fluid isolated from the drug reservoir, as desired.

The infusion pump 10 includes an elastomeric sleeve 110 surrounding a support member 112 and inlet and outlet ports 114, 116, respectively. In one particular embodiment, the support member 112 is cylindrical, but other shapes may be used, as desired. The elastomeric sleeve 110 is expandable in a radial direction about the support member 112. An inlet 114, connecting with a channel in the support member 112, is used to fill the elastomeric sleeve 110 with an impelling fluid 119, thus expanding the elastomeric sleeve 110. In one particular embodiment, the inlet 114 includes a one-way valve (not shown) to prevent fluid 119 within the sleeve 110 from exiting back through the inlet 114. The inlet 114 can be sealed with a cap 115. If desired, the pump 10 can be made, configured and operate as described in connection with the infusion pump of U.S. Pat. No. 7,322,961, incorporated herein by reference.

Once expanded, the elastomeric sleeve 110 exerts a pressure on the impelling fluid 119 contained therein. The pump 10 additionally includes an outlet 116 in fluid communication with channels 117, 118 permitting a pressurized stream of impelling fluid 119 to flow in the direction of arrow “A” and into the interface adapter 20, when a force is applied to the sleeve 110. A connector (not shown) is provided external to the outlet 116. In one particular embodiment, the connector is a luer lock connector.

The interface adapter 20 is connected at a first side 22 to the outlet 116 of the pump 10 by a mating, fluid-tight connector, which in one particular embodiment is a luer lock connector 122 mating with the luer lock connector of the outlet 116. At its other end 24, the interface adapter 20 connects to the drug reservoir 30 with a fluid-tight mechanical connector 124. In particular embodiments, the connector 124 may be a screw lock or a tongue-and-groove lock connector, as desired. A lumen of an impelling chamber 120 is connected between the connectors 122 and 124. The length, volume and cross-sectional area of the lumen of the impelling chamber can be configured to impose a desired pressure and/or rate of flow on the impelling fluid passing into and through the impelling chamber 120.

In one particular embodiment of the invention, the impelling fluid 119 of the interface adapter 20 is fluidly isolated from the medicinal fluid 135 contained in the drug reservoir 30, so that the medicinal fluid 135 does not make contact with the impelling fluid 119, nor with the interface adaptor 20 or pump 10. More particularly, in the embodiment illustrated in FIG. 2, the impelling fluid 119 of the interface adapter 20 is fluidly isolated from the medicinal fluid 135 contained in the drug reservoir 30 by a rubber gasket or plunger gasket 132 of a syringe 130 containing the drug reservoir 30. Although, in the present embodiment, the impelling fluid 119 will enter the emptied portions of the drug reservoir 30 as the gasket 132 pushes the medicinal fluid 135 out from the reservoir 30, the impelling fluid 119 does not come into contact with the medicinal fluid 135 itself.

The rubber gasket 132 is configured as a plunging element to plunge the medicinal fluid 135 from the reservoir 30 in response to a driving force pushing (i.e., moving) the gasket 132 from one end of the reservoir 30 towards the other end. The gasket 132 has an outer diameter that closely approximates the inner diameter of the drug reservoir 30 of the syringe 132, so as to permit movement within the drug reservoir 130 without permitting impelling fluid and medicinal fluid to mix on either side of the gasket 132. In other words, although the impelling fluid 119 will enter the syringe 130 on one side of the gasket 132, it does not pass through to the other side of the gasket 132. The gasket 132 thus acts as an element, isolating the medicinal fluid 135 from the impelling fluid 119, while driving the medicinal fluid 135 from the reservoir 30.

In use, medicinal fluid 135 is driven out of the drug reservoir 30 of the syringe 130 when a force applied to the elastomeric sleeve 110 forces pressurized impelling fluid out of the sleeve 110, through the impelling chamber 120 and into the syringe 130 on one side of the rubber gasket 132. The pressurized fluid 119 forced from the impelling chamber 120 thus drives the rubber gasket 132 from one end (i.e., the filled position) of the drug reservoir 30 to the other end (i.e., the empty position), and drives the medicinal fluid 135 from the outlet 137 and into the patient 40, via a tubing set or other delivery device 32. The gasket 132, thus driven, both empties the medicinal fluid 135 from the drug reservoir 30 and isolates the medicinal fluid 135 from the impelling fluid 119. In this way, the pump 10 and interface adapter 20 do not come into fluidic contact with the medicinal fluid 135, thus eliminating chemical incompatibility between the medicinal fluid 135 and the sleeve 110, and/or leaching of the container material from the pump 10 into the medicinal fluid 135.

Although illustrated as being contained in a syringe 130, it should be understood that the drug reservoir 30 can configured in any shape and from any material desired, including but not limited to, a regular syringe, a prefilled syringe, a glass syringe, a drug vial and/or a bag or similar reservoir, without departing from the scope of the present invention.

Referring now to FIGS. 1 and 3, there is shown another particular embodiment of the invention. Note that, with regard to FIGS. 2 and 3, like reference numerals will be used to denote like components, except that a prime (′) is added in connection with the impelling chamber 120′ of FIG. 3. More particularly, the embodiment of FIG. 3 is substantially similar to that of FIG. 2, with the main different being that, in the present embodiment of FIG. 3, the interface adapter 20 includes a tight cap 124 at one end of the drug reservoir 30, that connects to a balloon 128 at one end of the impelling chamber 120′, distal from the pump 10. The balloon 128 is inflated under pressure by the impelling fluid 119 forced from the sleeve 118 and into the impelling chamber 120′. As it inflates, the balloon 128 pushes against the rubber gasket 132 at the end of the lumen of the drug reservoir 30. Note that, in the present embodiment, impelling fluid 119 does not actually touch the walls of the drug reservoir 30 as the gasket 132 is moved through the reservoir, in contrast to the embodiment of FIG. 2. Rather, in the present embodiment, the impelling fluid 119 is fully contained within the balloon 128. The balloon 128 and/or the gasket 132 act as an isolation element, isolating the medicinal fluid 135 from the impelling fluid 119, while simultaneously forcing the medicinal fluid 135 out of the outlet 137 of the drug reservoir 30 and into a tubing set or other delivery device 32.

In one further alternate embodiment, not shown, the gasket 132 is omitted and the walls of the balloon 128 are used to isolate the impelling fluid 119 from the medicinal fluid 135 and to drive the fluid 135 from the chamber 30, as the balloon 128 is inflated into and through the chamber 30. Thus, in the present embodiment, the balloon 128 acts as an isolation element, isolating the medicinal fluid 135 from the impelling fluid 119, while driving the medicinal fluid 135 from the reservoir 30.

Referring back to FIG. 1, although the foregoing embodiments are described in connection with an elastomeric pump, the invention is not limited only thereto. Rather, the present invention can also be implemented using a pump 10 other than an elastomeric pump to deliver the force to the impelling fluid, without departing from the scope or spirit of the present invention. For example, in accordance with one particular embodiment of the invention, the pump 10 includes an electronic valve operable to connect an interface adapter 20, 20′ to a miniature pump that pumps an impelling fluid 119 into the lumen of the interface adapter 20, 20′, in order to drive the gasket 132 through a drug reservoir 30. Similarly, the pump 10 can include a peristaltic pump, a spring driven pump, a shaft driven by a motor, etc., as desired.

More particularly, a non-elastomeric pump 10 would operate in the same manner as described in connection with the elastomeric pump 10 of FIGS. 2 and 3, to provide a force to the impelling fluid, forcing the impelling fluid through the interface adapter 20 to drive the medicinal fluid from the drug reservoir 30 into the patient 40, while isolating the impelling fluid from the medicinal fluid in the reservoir 30.

As can be seen from the foregoing, the present invention provides a system including a pump wherein at least some of the components are reusable. An impelling fluid is used to drive the medicinal fluid from a drug reservoir, without interacting with the medicinal fluid. Similarly, in one particular embodiment, the pump of the system is operated without its elastomeric or other pump elements interacting directly with the medicinal fluid. This provides a cost savings by permitting reuse of the pump components, and additionally advantageously prevents leaching from the pump components into the medicinal fluid.

The present invention provides an improved pump apparatus, system and method of use as described herein. Accordingly, while a preferred embodiment of the present invention is shown and described herein, it will be understood that the invention may be embodied otherwise than as herein specifically illustrated or described, and that within the embodiments certain changes in the detail and construction, as well as the arrangement of the parts, may be made without departing from the principles of the present invention as defined by the appended claims.

1. A system for delivering a medicinal fluid, comprising:
a pump including a fluid outlet and an elastomeric sleeve, said elastomeric sleeve configured to inflate in response to the receipt of an impelling liquid in said elastomeric sleeve, said elastomeric sleeve additionally configured to discharge pressurized impelling fluid from said elastomeric sleeve via said fluid outlet;
a drug reservoir including a medicinal fluid;
an isolation element connected between said fluid outlet of said pump and said medicinal fluid of said drug reservoir;
said isolation element configured to drive medicinal fluid from an outlet opening of said drug reservoir in response to a force applied to said isolation element by pressurized impelling fluid exiting said pump fluid outlet, said isolation element fluidly isolating said medicinal fluid from said impelling fluid.

2. The system of claim 1, wherein the isolation element includes a rubber gasket disposed in a lumen of said drug reservoir.

3. The system of claim 2, wherein the isolation element further includes a balloon in contact with one said of said rubber gasket, said balloon inflatable by said pressurized impelling fluid.

4. The system of claim 1, wherein said isolation element includes a balloon inflatable by said pressurized impelling fluid.

5. The system of claim 1, wherein said pump is separated from said drug reservoir by an interface adapter including an impelling chamber configured to provide pressurized impelling fluid against said isolation element.

6. The system of claim 1 wherein the drug reservoir is part of a syringe and said isolation element includes a gasket or plunging element of said syringe, one side of said syringe being in fluid communication with a medicinal fluid of said drug reservoir, an opposite side of said gasket in fluid communication with said fluid outlet of said pump.

7. The system of claim 6, wherein said gasket or plunging element is separated from said fluid outlet by an interface adapter.

8. The system of claim 7, wherein said interface adapter includes an impelling chamber having at least one of a length, volume or cross-sectional area selected to impose a desired pressure or rate of flow on the impelling fluid passing through said impelling chamber.

9. The system of claim 1, wherein said pump additionally includes a fluid inlet, separate from said fluid outlet, in fluid communication with said elastomeric sleeve.

10. The system of claim 1, further including water as the impelling fluid.

11. A pump device for delivering a medicinal fluid, comprising:
a pump for driving an impelling fluid under pressure;
a drug reservoir configured to receive a medicinal fluid;
an isolation element fluidly isolating pressurized impelling fluid driven by said pump from medicinal fluid received in said drug reservoir; and
said isolation element configured to drive said medicinal fluid from said drug reservoir in response to a force applied to said isolation element by impelling fluid pumped from said pump.

12. The pump device of claim 11, wherein said drug reservoir is contained in a syringe in fluid communication with an output of said pump.

13. The pump device of claim 12, wherein said isolation element includes a balloon.

14. The pump device of claim 12, wherein said isolation element includes a rubber gasket.

15. The pump device of claim 12, wherein said pump is separated from said syringe by an interface element including an impelling chamber.

16. The pump device of claim 15, wherein at least one of a length, volume or cross-sectional area of said impelling chamber is selected to impose a desired pressure or rate of flow on the impelling fluid passing through said impelling chamber.

17. The pump device of claim 11, wherein said impelling fluid is water.

18. A method of delivering a medical fluid to a patient, comprising the steps of:
providing a pump device according to claim 1;
filling the elastomeric sleeve with an impelling fluid that is not the medical fluid to be delivered;
applying a force to the elastomeric sleeve to drive pressurized impelling fluid against the isolation element with sufficient force to move the isolation element within the drug reservoir, movement of the isolation element within the drug reservoir pushing the medical fluid from an outlet of the drug reservoir.

19. The method of claim 18, wherein the impelling fluid is water.

20. The method of claim 18, wherein the isolation element includes a balloon.
